(19) United States
(12) Patent Application Publication
(10) Pub. No.: US 2010/0071708 A1
(43) Pub. Date: Mar. 25, 2010

(54) NOVEL HEARING PROTECTION DEVICE AND METHOD AND SECURE EARBUD ASSEMBLY

(76) Inventor: Martin L. Lenhardt, Hayes, VA (US)

Correspondence Address: Hershkovitz & Associates, LLC, 2845 Duke Street, Alexandria, VA, 22314, US

(21) Appl. No.: 12/522,161

(22) PCT Filed: Jan. 4, 2008
(86) PCT No.: PCT/US08/50243

§ 371 Date: Jul. 3, 2009

Related U.S. Application Data
(60) Provisional application No. 60/878,364, filed on Jan. 4, 2007.
(60) Provisional application No. 60/878,368, filed on Jan. 4, 2007.

(51) Int. Cl. A61F 11/08

(52) U.S. Cl. 128/868; 181/135

(57) ABSTRACT

A passive level dependent hearing protection device for insertion into an ear canal is made of a flexible bladder attached to a frame and where the bladder utilizes valves to evacuate air from the ear canal. The device thereby causes transmission loss of acoustic waves wherein low frequency steady state and impact noises are attenuated but soft noises such as speech are attenuated to a lesser degree. The device incorporates a safety feature for preventing creation of too low an air pressure in the ear canal. Finally, the device may incorporate a transducer.

### CROSS-REFERENCE TO RELATED APPLICATIONS

This application claims the benefit of provisional patent application No. 60/878,364 entitled “PUMPPLUG-NOVEL HEARING PROTECTION DEVICE AND METHOD” filed Jan. 4, 2007, the entirety of which is incorporated by reference. This application also claims the benefit of provisional patent application No. 60/878,368 entitled “SECURE EARBUD ASSEMBLY FOR LISTENING” filed Jan. 4, 2007, the entirety of which is incorporated by reference.

### FIELD OF INVENTION

This invention relates generally to a device and method of using said device for the protection of a user's hearing and also a secure means of retaining the device in an ear canal.

### BACKGROUND OF THE INVENTION

Noise-induced hearing loss (“NIHL”) is a debilitating condition that generally develops slowly and can severely affect an individual's ability to communicate. In some instances, however, hearing loss can be acute, especially when an individual is subjected to intense noises such as those occurring from the discharge of weaponry or concussive noises such as those from explosions. Additionally, the acute aftereffects of exposure to such noises can critically affect an individual's ability to function because of ear-ringing or other symptoms. For law-enforcement personnel or combat operatives, such affects can cause injury or the loss of life.

The National Institute for Occupational Safety and Health (“NIOSH”) suggests exposure to impulsive noises not exceed 140 dB SPL in one event per day. Because firearm discharges can range in intensity from about 140 dB to upwards of 170 dB, the likelihood of incurring NIHL for military or police personnel can be high. Some common means of preventing NIHL require a user to wear earmuffs, sometimes in addition to earplugs. While these devices can adequately protect a user's hearing, they also dampen critical sounds that would not result in NIHL. For example, a user would be protected from the NIHL resulting from a concussive round detonating nearby but would be unable to receive orders from another user because verbal sounds are attenuated as well. In addition, the Occupational Safety Health Administration (OSHA) [Occupational Safety and Health Administration. Technical Manual, Washington, D.C.: U S Department of Labor, 2002] mandates hearing protection if the noise level is at or exceeds 90 dBA for eight hours. Hearing protection is usually instituted at noise levels of 85 dBA. Vocal conversation level is about 65 dBA.

Additionally, wind noise and other ambient sounds often affect the pleasure of listening to music via small transducers placed in the ear canals (earbuds) due to masking and distraction. The typical listener strategy is to increase the music volume, often at the risk of hearing loss to intense sound exposure. Additionally, the off-centre weight of earbuds eventually causes many to be dislodged from the ear canals, most frequently during vigorous exercise when the canal geometry changes in response to jaw movements.

As such, there is a need for a device which allows a user to hear critical noises such as speech but which also allows a user to be protected from noises that would cause NIHL. In addition, the device should function to retain its position in the ear canal under various conditions, including exercise.

### SUMMARY AND OBJECTS OF THE INVENTION

A passive inexpensive level dependent hearing protection device (LDHPD) is described that suits military and police personnel's need for hearing protection with minimal interference with speech communication. The single-unit device is adapted to be simple to use and comfortable over hours of use. Therefore, in one embodiment of the present invention, a passive level dependent hearing protection device for insertion into an ear canal is described comprising (a) a flexible bladder attached to a frame, said frame adapted to fit within the ear canal; (b) at least one one-way valve on said bladder adapted to release air; (c) at least one one-way valve on said frame adapted to help evacuate air from the ear canal; (d) a valve adapted to crack, to release air pressure, and/or to crack and release air pressure when a critical low air pressure condition in the ear canal is reached; and (e) a plug attached to said frame, said plug adapted to provide a seal when said plug is inserted into the ear canal. Further, various embodiments of the present invention also comprise a method of using the device. The device is used by (a) inserting said plug into the ear canal; (b) squeezing said bladder; (c) allowing said bladder to evacuate air from the ear canal by releasing the squeezing pressure on said bladder; and (d) removing said device when hearing protection is no longer needed. In another embodiment, the device may be inserted after the bladder has been squeezed but before the squeezing pressure has been removed. The device also incorporates a valve that releases the negative air-pressure when a set negative air-pressure is reached. The valve can accomplish this by “cracking”. Cracking is defined as encompassing the valve moving to an open state, the valve bursting, or any other movement which releases air into the vacuum created by the bladder. Finally, in an alternate embodiment, the device is adapted with a transducer and may or may not contain an aperture.

### BRIEF DESCRIPTION OF THE DRAWINGS

The present invention can best be understood in connection with the accompanying drawings. It is noted that the invention is not limited to the precise embodiments shown in drawings, in which:

FIG. 1 is a cross-sectional view of one embodiment of the present invention;

FIG. 2 is a diagram illustrating insertion of the device;

FIG. 3 is a diagram illustrating the device inserted in an ear;

FIG. 4 is a cross-sectional view of the device being activated;

FIG. 5 is a cross-sectional view of the device evacuating air from the ear canal;

FIG. 6 is a cross-sectional view of the device after activation and air evacuation in an ear; and

FIG. 7 is a cross-sectional view of the device containing a transducer but no open-air aperture.

### BRIEF DESCRIPTION OF REFERENCE NUMERALS

50 Pump-Plug Hearing Protection Device; 100 Orifice; 102 Flexible Bladder; 104 Frame; 106 Closed Plug; 108 Silicone Valve; 110 One-Way Valve (Bladder); 112 One-Way Valve (To Ear-Canal); 114 Aperture; 200 Pinna; and 500 Transducer.

### DETAILED DESCRIPTION OF THE INVENTION

The current invention, in a preferred embodiment, provides protection from impact sounds by creating a negative pressure between the plug tip and the eardrum (preferably around −100 mm H₂O or −1 k Pa). Squeezing the peripheral plug end, thereby evacuating a fixed amount of air from the canal, creates the negative pressure. A miniature one-way valve will maintain the pressure with an overpressure override for safety. In effect the “pump plug” will have a similar acoustical action on the eardrum as a normal stapedius muscle contraction, but without its limitations (too slow and fatigue) in the presence of impulse noise. The plug cannot be vented, as with the current U S Army plugs; however, because the venting attenuation reduction effect will be accomplished in addition to using a very compliant valve mechanism, passive transmission of mid and high frequency acoustics can occur through the presence of a small aperture passage. The dimensions of the device may be variable to allow for perfect fit in a variety of ear canals according to the dimensions of a specific user.

Again, the design principle is that a low negative pressure can be comfortably created in the canal which will produce the desired attenuation but which will also protect against low frequency steady state and impact noise. The valve system will act as a compliant window, much like a small air opening (venting). Further, a small aperture will connect the valve to ambient noise and pressure. The most effective aperture diameter, and thereby a preferred aperture diameter for optimal sound transmission, appears to be 0.010″. Other sizes may be employed. Thus the design will allow soft sounds, especially speech, to remain soft, but intense sounds will be attenuated. The LDHPD can be tunable in that more negative pressure can be applied depending on the bladder size of the plug. Wind noise is expected to be minimal due to the vacuum effect created by the bladder. Because the device is simple, only water cleaning is necessary to maintain the device.

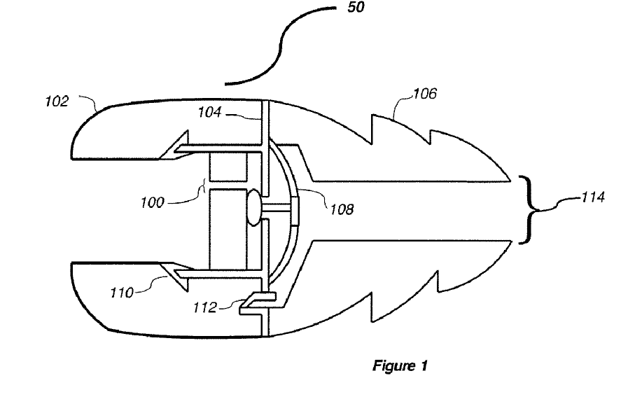

The device can be best understood diagrammatically. Turning now to the figures, FIG. 1 shows a cross-sectional view of one embodiment of the present invention 50. The aperture (or orifice) 100 allows sound transmission to the overpressure valve 108. The overpressure valve may be any material such as plastic but in one preferred embodiment it is comprised of silicone. The large aperture 114 then transmits sound to the ear canal. Surrounding the large aperture is the closed plug 106, which is adapted to snugly fit within the ear canal and may be comprised of any standard material including urethanes, polyurethanes, plastics, or other compliant materials. The bladder 102 is used to evacuate pressure from the ear canal. When squeezed, the bladder 102 is evacuated of air through at least one one-way valve 110. The negative air-pressure created in the bladder then pulls air from the ear canal through at least one other second one-way valve 112 until the bladder's air pressure is equalized. Valve 108 can have a variety of overpressure limits such as −1, −2, or −3 kilopascals (kPa) or some other pressure depending on the attenuation effect desired. Finally, frame 104 comprises the foundation of the device by providing a structure for the bladder, plug, and valves to be constructed about. The frame can be made from any material, but plastics such as urethane and/or polyurethane are preferred because they are cost-effective.

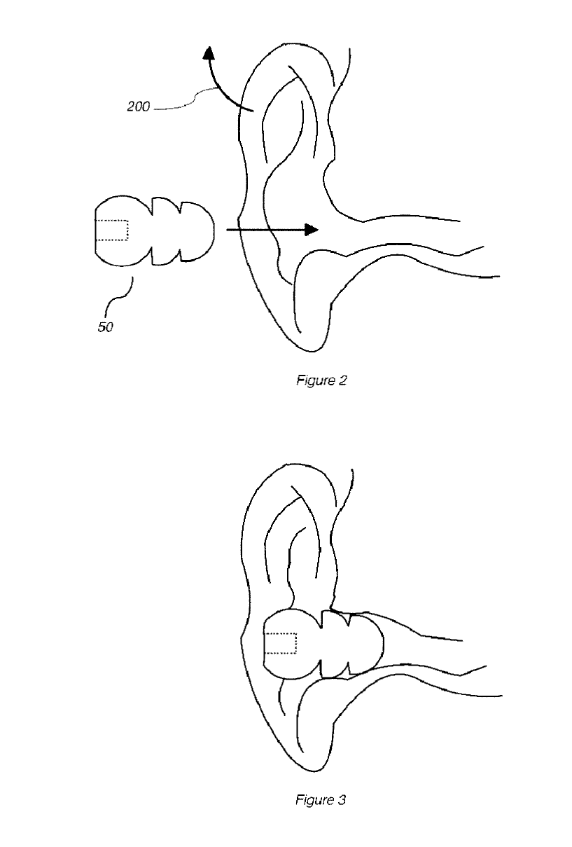

FIG. 2 shows the device 50 being inserted into the ear. The pinna 200 of the ear may be pulled to aid insertion but this is not necessary. FIG. 3 shows the device inserted in an ear.

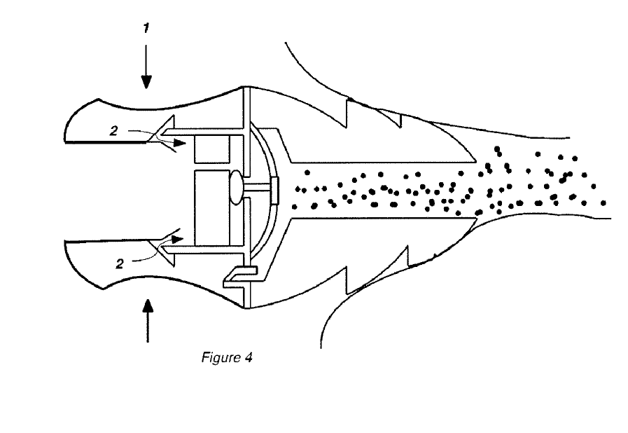

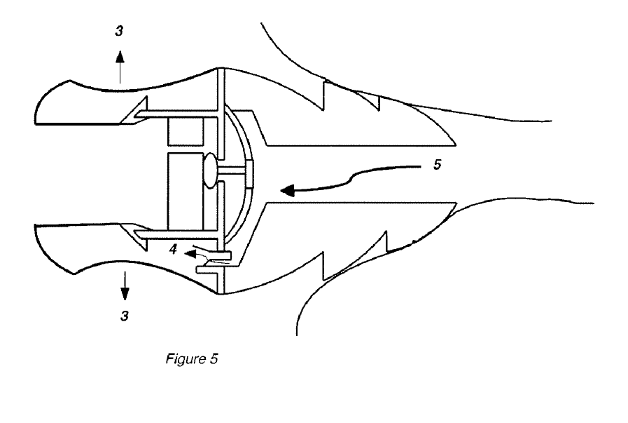

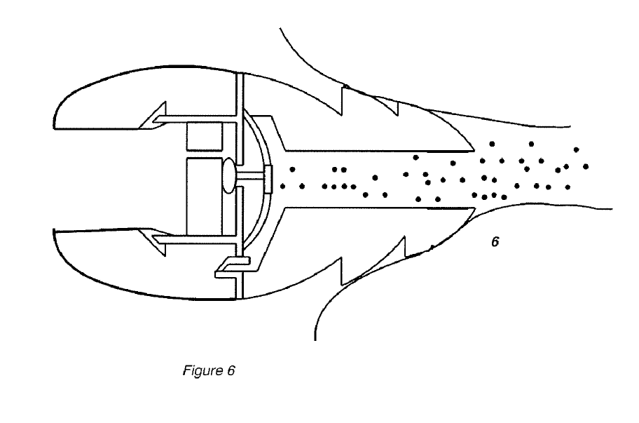

FIG. 4 shows the device wherein the device is squeezed 1 and air pressure is released 2. FIG. 5 shows the squeezing pressure released 3, air pressure entering into the bladder 4 from the ear canal 5. FIG. 6 shows the steady state of the device after the air pressure in the ear canal has been reduced.

With a negative pressure of −2 kPa the transmission loss is ˜20-25 dB. It is estimated that a negative pressure of −1 kPa (the preferred target for the proposed vacuum plug) would result in a transmission loss of about 15 dB. The vacuum effect is maximal in low frequencies for which noise from, e.g., low caliber arms fire will be reduced. The vacuum effect also maintains the earplug in the ear even with movement of the lower jaw, e.g., as in eating, etc. The vacuum also keeps the plug from moving as a result of sound exposure on the distal end. Without vacuum, earplugs loose their “seating” from the effects of sound pressure and become less effective, usually requiring re-insertion.

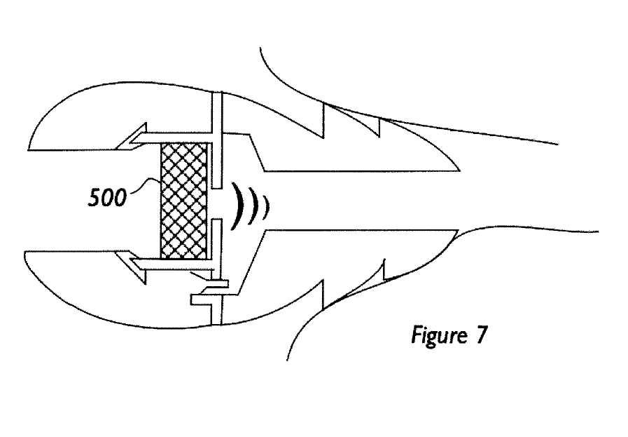

In an alternate embodiment, the ear assembly can be modified to include a transducer. In one sub-embodiment, the device functions to limit impact and other high intensity noises while also providing a transducer for audio signals. This would find application, for example, in battlefield communication applications, such as for transmitting a communication to a soldier in a high-noise environment. The transducer may be situated in a manner to retain the open-air aperture or, as in FIG. 7, the open-air aperture may be omitted.

In another sub-embodiment, the device can utilize a lower, i.e. closer to ambient, negative air-pressure, preferably around −1 kPa, to act as a securing means for the assembly into the ear. While impact noise would not be reduced to the same extent as at higher pressures, the device can be used for, e.g., recreational uses such as jogging or other activities in which the device might otherwise become dislodged without a securing means. The vacuum attenuates ambient noise (though to a lesser extent) and holds the earbud/plug in place, even during vigorous exercise. Squeezing the plug end, evacuating a fixed amount of air from the canal, will create the negative pressure.

FIG. 7 shows one possible arrangement of a transducer/earbud assembly. Like the other embodiments of the present invention, the device uses a bladder to evacuate air from the ear-canal. The device, in this embodiment, no longer utilizes an open aperture to allow sound signals to pass, since the focus of the assembly is to transmit the signal from the transducer 500 and not the environment. The body of the plug may be made of urethane, polyurethane or other materials including polypropylene. The flanged design allows for comfortable and secure insertion into the ear canal. Note, the transducer may be a high-fidelity audio transducer but may also comprise non-traditional transducers such as those emitting signals in the ultrasonic range. The device may be made waterproof for use in the rain or during swimming.

In the foregoing description, certain terms and visual depictions are used to illustrate the preferred embodiment. However, no unnecessary limitations are to be construed by the terms used or illustrations depicted, beyond what is shown in the prior art, since the terms and illustrations are exemplary only, and are not meant to limit the scope of the present invention. It is further known that other modifications may be made to the present invention, without departing the scope of the invention, as noted in the appended claims.

I claim:

1. A passive level dependent hearing protection device for insertion into an ear canal comprising:
a. a flexible bladder attached to a frame, said frame adapted to fit within the ear canal;
b. at least one one-way first valve on said bladder adapted to release air;
c. at least one one-way second valve on said frame adapted to help evacuate air from the ear canal;
d. a third valve adapted to crack, to release air pressure, and/or to crack and release air pressure when a critical low air pressure condition in the ear canal is reached; and
e. a plug attached to said frame, said plug adapted to provide a seal when said plug is inserted into the ear canal.

2. The device of claim 1 in which the frame comprises polyurethane materials.

3. The device of claim 1 in which said device has an aperture surrounded by said bladder which leads to said third valve.

4. The device of claim 3 in which said third valve is made of silicone.

5. The device of claim 3 in which said second aperture has a diameter of about 0.010 inches.

6. The device of claim 1 in which said bladder is adapted in size to adjust the decrease in air pressure produced in the ear canal when the device is used.

7. The device of claim 1 in which the plug is comprised of urethane.

8. The device of claim 1 in which said third valve is adapted to release at an overpressure of around −1 kilopascal.

9. The device of claim 1 in which said third valve is adapted to provide a transmission loss of around 20 to 25 decibels at a negative air pressure of around −2 kilopascals.

10. The device of claim 1 in which said third valve is adapted to provide a transmission loss of around 15 decibels at a negative air pressure of around −1 kilopascal.

11. The device of claim 1 in which said device provides a vacuum which prevents movement of said device in said ear canal.

12. A method of using the device in claim 1 comprising the steps of:
a. inserting said plug into the ear canal;
b. squeezing said bladder;
c. allowing said bladder to evacuate air from the ear canal by releasing the squeezing pressure on said bladder; and
d. removing said device when hearing protection is no longer needed.

13. The method of 12 in which said plug is inserted while the pinna of the ear is retracted.

14. A method of using the device in claim 1 comprising the steps of:
a. squeezing said bladder;
b. inserting said plug into the ear canal;
c. allowing said bladder to evacuate air from the ear canal by releasing the squeezing pressure on said bladder; and
d. removing said device when hearing protection is no longer needed.

15. The method of 14 in which said plug is inserted while the pinna of the ear is retracted.

16. A secure ear-bud assembly for insertion into an ear canal comprising:
a. a flexible bladder attached to a frame, said frame adapted to fit within the ear canal;
b. at least one one-way first valve on said bladder adapted to release air;
c. at least one one-way second valve on said frame adapted to help evacuate air from the ear canal;
d. a transducer adapted to produce an acoustical signal; and
e. a plug attached to said frame, said plug adapted to provide a seal when said plug is inserted into the ear canal.

17. The assembly of claim 16 further comprising an aperture.

18. A method of using the assembly of claim 16 comprising the steps of:
a. squeezing said bladder;
b. inserting said plug into the ear canal squeezing;
c. allowing said bladder to evacuate air from the ear canal by releasing the squeezing pressure on said bladder; and
d. removing said device when hearing protection is no longer needed.
